(19) United States
(12) Patent Application Publication
(10) Pub. No.: US 2013/0211132 A1
(43) Pub. Date: Aug. 15, 2013

(54) METHOD FOR PREPARING DI-ALPHA-METHYL BENZYL ADIPATE

(71) Applicant: Edson Rodrigues, Pirassununga (BR)

(72) Inventor: Edson Rodrigues, Pirassununga (BR)

(73) Assignee: RHODIA POLIAMIDA E ESPECIALIDADES LTDA, Sao Paolo (BR)

(21) Appl. No.: 13/819,693

(22) PCT Filed: Aug. 17, 2011
(86) PCT No.: PCT/IB2011/001900

§ 371 Date: Apr. 30, 2013

(30) Foreign Application Priority Data
Aug. 27, 2010 (FR) 1056802

(51) Int. Cl. C07C 67/08

(52) U.S. Cl. CPC C07C 67/08
USPC 560/193

(57) ABSTRACT

A method for preparing di-alpha-methyl benzyl adipate by direct esterification of adipic acid and alpha-methylbenzyl alcohol is described. The reaction can produce a high yield and can be carried out under mild conditions, in the presence of a catalyst selected from among the orthotitanates of tetra-hydrocarbyl.

Di(α-methylbenzyl) adipate or styrallyl adipate is known in the art as plasticizer for polymers (for example for polyvinyl acetate and polyvinyl chloride, or chlorinated or chlorosulfonated polyethylene) but it also contributes to improving other functions, such as the processability (for example for styrene/acrylonitrile copolymers, acrylonitrile/butadiene/styrene resins, methyl methacrylate/butadiene/styrene resins), the fluidity (for example for the molding of polystyrene), low temperature properties and ozone-resistance properties (for example for chlorinated or chlorosulfonated polyethylene vulcanizate).

The prior art discloses different ways of preparing di(α-methylbenzyl) adipate:
U.S. Pat. No. 2,755,262—provides two methods for the preparation of the di(α-methylbenzyl) ester: (i) by reaction of α-methylbenzyl alcohol with the dibasic acid chloride in the presence of a tertiary base, such as dimethylaniline, pyridine or quinoline, for the purpose of the reaction with the hydrogen chloride byproduct, (ii) by ester exchange between the α-methylbenzyl alcohol and the dimethyl or diethyl ester of the dibasic acid, in the presence of a metal alkoxide as catalyst.
Zeinalov B. K. et al. (Synthesis of α-phenylethyl esters of dibasic aliphatic acids, and a study of their plasticizing properties, in Sin. Prevrashch. Monomernykh Soedin, 1967, pages 157-161, Baku, USSR) synthesized di(α-methylbenzyl) adipate by reaction of α-methylbenzyl chloride with adipic acid in the presence of triethylamine.
JP 46038692 shows the preparation of di(α-methylbenzyl) adipate by transesterification of α-methylbenzyl alcohol (styrallyl alcool—CAS 98-85-1) with an adipate. The synthesis used diethyl adipate and an α-methylbenzyl alcohol:diethyl adipate molar ratio=2.05. This synthesis was carried out in the presence of 0.6 part of sodium ethoxide per 37 parts of ethanol and uses toluene as solvent (the reactants were dissolved in 270 parts of toluene).
JP 46038693 reveals that di(α-methylbenzyl) adipate can be prepared by reaction of the carboxylic acid chloride with α-methylbenzyl alcohol or α-methylbenzyl chloride with the carboxylic acid.

Research on an improved process for preparing di(α-methylbenzyl) adipate is currently underway and is targeted at commercially viable production, with improved yields under mild conditions—which represents a reduction in the energy consumption—in order not to promote the formation of undesirable byproducts which require additional purification operations—which represents a reduction in the cost.

### DESCRIPTION OF THE INVENTION

The main subject matter of the present invention is a process for the preparation of di(α-methylbenzyl) adipate by the esterification of adipic acid and α-methylbenzyl alcohol in the presence of a tetrahydrocarbyl orthotitanate catalyst. The invention relates in particular to a batchwise or continuous process for the preparation of di(α-methylbenzyl) adipate by the direct catalytic esterification, in the presence of tetrahydrocarbyl orthotitanates, of adipic acid and α-methylbenzyl alcohol.

Despite the basic knowledge according to which carboxylic acid esters can be prepared by reaction of carboxylic acids with alcohols, the steric hindrance of the alcohol and of the carboxylic acid has always been regarded as an obstacle to this type of esterification. The Applicant Company has been able to overcome the preconceptions resulting from the prior art and to obtain high conversion yields by virtue of a specific combination of the esterification conditions with a judicious choice of the selective catalysts, such as tetrahydrocarbyl orthotitanates, which promote the reaction and which at the same time help to prevent the formation of secondary products, for example styrene and the corresponding ether, during the esterification.

A specific embodiment of the process of the invention comprises the following stages:

a. in a molar ratio of the α-methylbenzyl alcohol to the adipic acid of 2.0:1 to 10:1, the α-methylbenzyl alcohol corresponding to a molar excess of 10% to 20% with respect to the adipic acid, and a tetrahydrocarbyl orthotitanate catalyst were charged to an apparatus having a reaction vessel with a condensation column and having stirring means;
b. The mixture was heated up to approximately 180° C.
c. The water/alcohol azeotrope formed was continuously withdrawn, in particular up to an acidity of less than 20 mg KOH/g, more particularly between 10 and 5 mg KOH/g;
d. The excess α-methylbenzyl alcohol was withdrawn;
e. The unreacted adipic acid was neutralized and washed out.

The process of the invention can optionally subsequently comprise the following independent stages, which are independent of one another:
the residual α-methylbenzyl alcohol was removed, in particular by steam stripping;
the remaining product was dried;
the remaining product was filtered.

The process of invention is suitably carried out continuously or batchwise, in particular batchwise.

Optionally, the dissolved oxygen is withdrawn from the α-methylbenzyl alcohol, before it is mixed with the acid and the catalyst, and the heating of the mixture in the reaction vessel, and/or the adipate obtained is subjected to cleaning for the purpose of the suppression of the color of the reaction product. In a specific embodiment, all or a portion of the esterification reaction is carried out under an inert atmosphere. For example, a slight stream of nitrogen is employed during the heating of the reactants up to 120° C., until the acid is dissolved in the alcohol.

In a specific embodiment, the catalyst is predissolved in a sufficient amount of alcohol, before being added to the mixture of alcohol and acid. Also specifically, the mixture of α-methylbenzyl alcohol and adipic acid is heated to the reaction temperature, preferably to approximately 180° C., before the addition of the catalyst.

The suitable tetrahydrocarbyl orthotitanates correspond to the formula Ti(OR)₄, in which R is a cyclic, linear or branched aliphatic hydrocarbon radical comprising from 1 to 24 carbon atoms, in particular from 1 to 6 carbon atoms. Mention may be made, as suitable orthotitanates, without this list being limiting, of tetramethyl, tetraethyl, tetra(n-butyl), tetraisobutyl, tetra(sec-butyl), tetra(tert-butyl), tetraisopropyl, tetraoctadecyl, tetraphenyl, tetra(n-pentyl), tetra(n-pentenyl) and tetra(n-hexyl) titanates. An appropriate amount of this catalyst is between 10⁻⁶% and 1%, in particular between 0.001% and 0.003%, of the weight of the mixture of adipic acid and α-methylbenzyl alcohol.

In particular, although this is not obligatory, the esterification process of the invention does not use entrainers to extract the water formed from the reaction vessel in order to displace the thermodynamic equilibrium towards the adipate. Instead of this, the temperature used in the reaction promotes the formation of a binary azeotrope between the α-methylbenzyl alcohol (11%) and the water (89%), with a boiling point at 93.5° C., making it possible for the azeotrope to be extracted from the reaction vessel in order to remove the water formed. The densities of the phases formed after cooling are very similar, thus difficult to separate, so a molar excess of alcohol, preferably from approximately 10% to approximately 20%, is preferably used, with respect to the stoichiometric requirements, and the alcohol withdrawn is not recycled in the reactor.

An α-methylbenzyl alcohol/adipic acid molar ratio which is suitable for the reaction is from 2.0:1 to 10:1, in particular from 2.0:1 to 10:2, in particular from 2.4:1 to 2.8:1. The α-methylbenzyl alcohol content can represent an excess of 10 to 20 mol %, with respect to the stoichiometric amount.

Also in a specific embodiment, when the reaction reaches a conversion of 70%, the amount of water produced by the reaction decreases and thus the amount of azeotrope is also reduced. Thus, the pressure is reduced, in order to maintain the temperature of the reaction mixture above the boiling point. Preferably, the reduction in the pressure is slow, down to 20 mmHg.

The excess alcohol is removed in particular at approximately 180° C. under 20 mmHg, and the alcohol withdrawn is no longer recycled in the reactor.

The stages used for the preparation of the di(α-methylbenzyl) adipate, after the esterification stage, are described in detail below:

a. The unreacted adipic acid is removed by neutralization and washing. For example, an aqueous sodium carbonate solution is added to the resulting solution at 50-55° C. in order to neutralize an unreacted acid and to hydrolyze the catalyst. The use of a strong alkaline solution, for example of sodium hydroxide or potassium hydroxide, is less preferred as it is more likely to bring about the hydrolysis of the di(α-methylbenzyl) adipate, making it more difficult to neutralize the acidity to a level acceptable in the case of plasticizers. After neutralization of the residual acidity, the aqueous phase is withdrawn from the reaction mixture and the reaction product is treated, for example by washing with a 10% by weight sodium chloride solution, in order to remove the residual monoesters.
b. The residual α-methylbenzyl alcohol is removed by steam stripping or in any other appropriate way.

Some unreacted residual alcohols, approximately 1-2% by weight, even after the withdrawal of the excess alcohol, generally remain in the organic phase during the neutralization and during the washing stages. If this is desirable, steam stripping at approximately 6.5 kgf/cm² can be used to remove the alcohol and a portion of the undesirable ether which has formed.

c. The yellowing of the ester can be prevented or suppressed by a cleaning stage.

A yellow color sometimes present in the adipate formed is probably related to the formation of impurities, the coloration of which is promoted or accelerated by the incorporation of oxygen during the esterification reaction. This can be considerably reduced by removing beforehand the oxygen dissolved in the starting alcohol, for example by sparging oxygen-free inert gas (for example nitrogen) into the alcohol before adding the acid and the catalyst and heating them. Alternatively, the slight yellowing of the final product which was acquired during the process can also be removed, for example by passing the final product through a filter containing active charcoal or by bringing the final product into contact with active charcoal and subsequently carrying out a filtration. The use of hydrogen peroxide is less preferred as, although it can reduce the yellowing, it can, on the other hand, increase the acidity.

The appropriate esterification temperature is approximately 180° C. Temperatures of approximately 190° C. and above promote the thermal decomposition of the di(α-methylbenzyl) adipate and the dehydration and condensation of the α-methylbenzyl alcohol.

The esterification stage itself is monitored in particular by titration of the reaction mixture, in order to determine its acidity, and/or by measurement of the water produced from the reaction mixture.

The optional drying of the final product is carried out in particular under low pressure, for example 70 mmHg, at a temperature of approximately 70° C., until a desired target moisture content is reached, for example of 0.2%, for example measured with a Karl Fischer apparatus.

### Example

The following embodiment of the present invention was carried out in an appropriate reactor having stirring means and a top condenser:
445 kg of adipic acid and 749 kg of styrallyl alcohol were charged to the reactor, at ambient temperature.
The temperature of the mixture was brought to 20° C., in the presence of a slight nitrogen stream, with stirring, until the acid had dissolved in the alcohol.
The temperature was then brought to 180° C., at ambient pressure, and 0.0267 kg of tetrabutyl titanate (Tyzor™ TNTB, sold by Dupont de Nemours), dissolved in the alcohol, was added.
At ambient pressure, the water/alcohol azeotrope was continuously withdrawn from the top of the condenser while the reaction was continued up to a conversion of 70%, and then the pressure was slowly reduced and maintained at approximately 20 mmHg, until the reaction mixture had reached a level of acidity of less than 20 mg KOH/g, more particularly between 10 and 5 mg KOH/g.
The temperature was reduced to approximately 55° C. and 19.6 kg of sodium carbonate in aqueous solution were added and mixed with stirring for 30 minutes, in order to neutralize the final product. The reaction product was subsequently washed with 13.1 kg of NaCl (10% by weight solution). The mixture was left standing, the phases then separated and the lower phase was withdrawn.
The remaining product was then subjected to steam stripping, at 6.5 kgf/cm² at 140° C. under a pressure of 30 mmHg, for 3 hours.
Finally, the final product was dried at a pressure of 40 mmHg, at 70° C., until the moisture content was approximately 0.2% by weight.

The yield of this process was 92% over a period of time of 40 h, and the degree of conversion was 96% over 24 h.

It is understood that the appended claims encompass separate embodiments of the invention, even those which are not expressly described in this text and which relate to equivalent functions in order to achieve equivalent results, which embodiments a person skilled in the art having average competence in the field concerned will be capable of implementing using the information and the example presented here.

1. A methods for the preparation of di(α-methylbenzyl) adipate, the method comprising conducting esterification of adipic acid and α-methylbenzyl alcohol in the presence of a tetrahydrocarbyl orthotitanate catalyst.

2. The method as defined by claim 1, wherein the tetrahydrocarbyl orthotitanate is selected from one or more tetramethyl, tetraethyl, tetra(n-butyl), tetraisobutyl, tetra(sec-butyl), tetra(tert-butyl), tetraisopropyl, tetraoctadecyl, tetraphenyl, tetra(n-pentyl), tetra(n-pentenyl) and tetra(n-hexyl) titanates.

3. The method as defined by claim 1, wherein the content of said tetrahydrocarbyl orthotitanate varies from 10⁻⁶% to 1%, in particular from 0.001% to 0.003%, by weight of the weight of the reactive mixture of adipic acid and α-methylbenzyl alcohol.

4. The method as defined by claim 1, wherein the α-methylbenzyl alcohol/adipic acid molar ratio varies from 2.0:1 to 10:1.

5. The method as defined by claim 1, wherein the α-methylbenzyl alcohol content represents an excess of 10 mol % to 20 mol %, with respect to the stoichiometric amount.

6. The method as defined by claim 1, wherein the process occurs in an apparatus comprising a reaction vessel having a condensation column and having stirring means, wherein the method comprises the following stages:
a. charging the α-methylbenzyl alcohol and the adipic acid in a molar ratio of 2.0:1 to 10:1, the α-methylbenzyl alcohol corresponding to a molar excess of 10% to 20%, and a hydrocarbyl orthotitanate catalyst;
b. heating the mixture up to approximately 180° C.;
c. continuously withdrawing the water/alcohol azeotrope formed, optionally up to an acidity of less than 20 mg/g KOH;
d. withdrawing the excess α-methylbenzyl alcohol; and
e. neutralizing and washing out the unreacted adipic acid.

7. The method as defined by claim 6, wherein the method it comprises the following stages, which are independent of one another:
a) removing the residual α-methylbenzyl alcohol, optionally by steam stripping;
b) drying the remaining product; and
c) filtering the remaining product.

8. The method as defined by claim 6, wherein the dissolved oxygen is withdrawn from the α-methylbenzyl alcohol before the reaction, optionally by sparging with an oxygen-free inert gas.

9. The method as defined by claim 6, wherein in stage c, when the conversion has reached 70%, the pressure is reduced.

10. The method as defined by claim 6, wherein all or a portion of the esterification reaction is carried out under an inert atmosphere.

11. The method as defined by claim 6, wherein the catalyst is predissolved in a sufficient amount of α-methylbenzyl alcohol, before being added to the mixture of alcohol and acid.

12. The method as defined by claim 6, wherein the mixture of acid and alcohol is heated, until the acid is completely dissolved in the α-methylbenzyl alcohol, before the catalyst is added to the mixture.

13. The method as defined by claim 6, wherein the mixture of α-methylbenzyl alcohol and adipic acid is heated to the reaction temperature, before the catalyst is added.

14. The method as defined by claim 6, wherein the removal of the excess α-methylbenzyl alcohol in stage d is carried out under low pressure.

15. The method as defined by claim 3, wherein the content of tetrahydrocarbyl orthotitanate various from 0.001% to 0.003%, by weight.

16. The method as defined by claim 4, wherein the molar ratio varies from 2.4:1 to 2.8:1.

17. The method as defined by claim 6, wherein the acidity is between 10 and 5 mg KOH/g.

18. The method as defined by claim 9, wherein the pressure is reduced to 20 mmHg.

19. The method as defined by claim 10, wherein the inert atmosphere is comprised of nitrogen.

20. The method as defined by claim 12, wherein the mixture of acid and alcohol is heated to 120° C.

21. The method as defined by claim 14, wherein the removal is carried out at a pressure of 20 mmHg.
